(19) United States
(12) Patent Application Publication
(10) Pub. No.: US 2008/0009806 A1
(43) Pub. Date: Jan. 10, 2008

(54) Blood sampling device

(75) Inventor: Jia-Ming Chang, Banciao City (TW)

Correspondence Address: RABIN & Berdo, PC, 1101 14TH STREET, NW, SUITE 500, WASHINGTON, DC, 20005, US

(73) Assignee: BIOTOP HOLDING CO., LTD.

(21) Appl. No.: 11/483,013

(22) Filed: Jul. 10, 2006

(51) Int. Cl. A61M 5/32

(52) U.S. Cl. 604/198

(57) ABSTRACT

A blood sampling device has a barrel, an inner tube, a base and a spring. The barrel has a front end, a rear end, a pipe and a cap. The pipe is formed on the front end. The cap is formed on the rear end. The inner tube is mounted in the barrel. The base is mounted in the inner tube to mount a needle hub and has multiple flexible tabs. The flexible tabs extend from the base and abut with the inner tube. The spring is mounted around the base. After pushing the cap into the rear end, the inner tube is moved toward the front end to squeeze the flexible tabs and the spring. Then, the base is retracted into inner tube automatically with the resilient force provided by the spring. Therefore, a user can be protected from being pricked by a used outer needle.

### BACKGROUND OF THE INVENTION

1. Field of the Invention

The present invention relates to a blood sampling device, and especially a safety blood sampling device for protecting a user from being pricked by a used needle.

2. Description of the Related Art

With modern developments in medical technology, people understand routes of infection are contamination of blood. Therefore, a blood sampling device requires safety to be operated.

A conventional blood sampling device comprises a barrel, a needle hub and a cap. The barrel comprises a front end. The needle hub is mounted in the barrel and comprises an outer needle and an inner needle. The outer needle protrudes on the front end of the barrel. After pricking a person with the outer needle, a vacuum tube inserts into the barrel and is pricked by the inner needle. After taking blood, a user covers a used outer needle with the cap to protect persons from being pricked by the used outer needle. However, the user may be pricked with the used outer needle during covering cap onto the used outer needle.

### SUMMARY OF THE INVENTION

The objective of the present invention is to provide a blood sampling device that can protect a user from being pricked by a used outer needle.

To achieve the foregoing objective, a blood sampling device has a barrel, an inner tube, a base and a spring. The barrel has a front end, a rear end, a pipe and a cap. The pipe is formed on the front end. The cap is formed on the rear end. The inner tube is mounted in the barrel. The base is mounted in the inner tube to mount a needle hub and has multiple flexible tabs. The flexible tabs extend from the base and abut with the inner tube. The spring is mounted around the base. After pushing the cap into the rear end, the inner tube is moved toward the front end to squeeze the flexible tabs and the spring. Then, the base is retracted into inner tube automatically with the resilient force provided by the spring. Therefore, a user can be protected from being pricked by a used outer needle.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a blood sampling device in accordance with the present invention;

FIG. 2 is an exploded perspective view of the blood sampling device in FIG. 1;

FIG. 3 is a side view in partial section of the blood sampling device in FIG. 1

FIG. 4 is an operational side view in partial section of the blood sample device in FIG. 1 during covering an opening with a cap; and

FIG. 5 is an operational side view in partial section of the blood sample device in FIG. 1 showing that a needle hub retracts into a barrel.

### DETAILED DESCRIPTION OF THE PREFERRED EMBODIMENTS

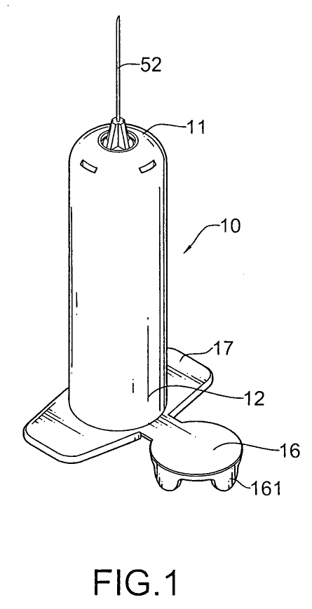

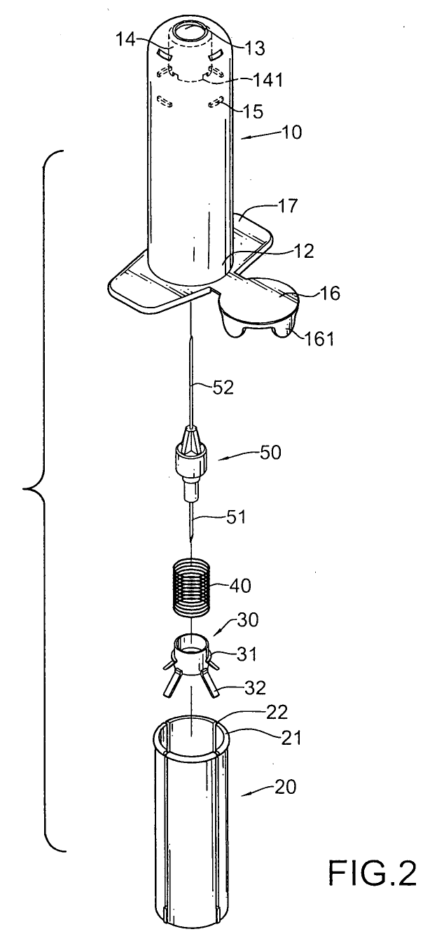

With reference to FIGS. 1 and 2, a blood sampling device in accordance with the present invention comprises a barrel assembly and a needle hub (50). The barrel assembly comprises a barrel (10), an inner tube (20), a base (30) and a spring (40). Preferably, the needle hub (50) may detach from the barrel (10) of the barrel assembly until the blood sampling device is in use.

The barrel (10) is hollow and comprises an inner surface, a front end (11), a rear end (12), a through hole (13), a pipe (14), at least one rib (15), an opening, a cap (16) and two optional finger flanges (17). The front end is closed and comprises an inner surface. The through hole (13) is defined through the front end (11). The pipe (14) is formed on the inner surface of the front end (11) and comprises multiple optional notches (141) and a free end. The notches (141) are defined in the free end. The at least one rib (15) is formed on the inner surface near the front end (11) and each one of the at least one rib (15) may be corresponding to each other. The opening is defined on the rear end. The cap (16) extends from the opening and comprises an inner surface, a circumference and at least one protrusion (161). The at least one protrusion (161) is formed around the circumference and on the inner surface. The finger flanges (17) respectively extend from the rear end (12).

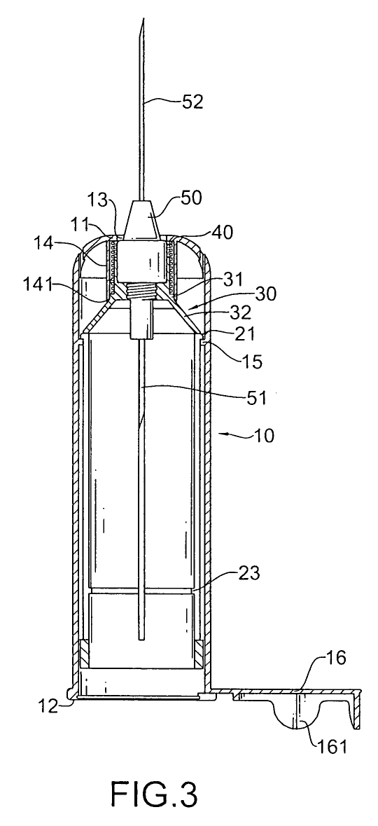

With further reference to FIG. 3, the inner tube (20) is movably mounted in the barrel (10) and comprises a front end, a rear end, an inner surface, an annular flange (21), multiple optional longitudinal grooves (22) and an optional stop (23). The annular flange (21) radially protrudes on the front end, comprises a bottom and abuts on the at least one rib (15). The bottom of the annular flange (21) abuts on the at least one rib (15). The longitudinal grooves (22) are defined through the inner tube (20) to make the front end resilient. The stop (23) protrudes on the inner surface near the rear end.

The base (30) is cylindrical, is mounted in the inner tube (20), and comprises a center through hole, at least one tab (31), multiple flexible tabs (32), a center, an outer circumference, an axis, an upper portion and an lower portion. The center through hole is defined in the center of the base (30). The at least one tab (31) protrudes on the outer circumference. The flexible tabs (32) extend from the lower portion, selectively abut with the annular flange (21) and may be corresponding to the notches (141).

The spring (40) is mounted around the upper portion of the base (30) and comprises two ends. One end of the spring (40) abuts against the inner surface of the front end (11) of the barrel (10). The other end of the spring (40) abuts against the tab (31) of the base (30).

The needle hub (50) is mounted in the base (30) and comprises an inner needle (51) and an outer needle (52). The inner needle (51) extends through the center through hole of the base (30). The outer needle (52) extends through the through hole (13) of the barrel (10).

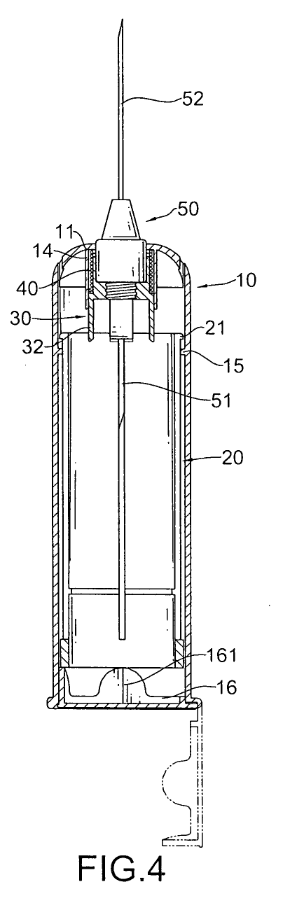

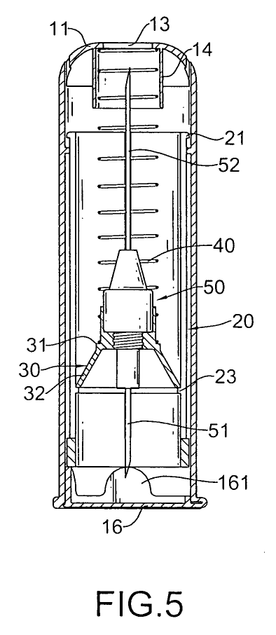

With further reference to FIGS. 3 to 5, after sampling blood and pressing the cap (16) into the opening of the rear end (12), the at least one protrusion (161) pushes the inner tube (20) to the front end (11). Then, the base (30) is moved toward the pipe (14) so the flexible tabs (32) are squeezed toward the axis of the base (30) by the pipe (14) to release from the annular flange (21). At the same time, the base (30) compresses the spring (40), and the force provided by the spring (40) will push the base (30) with the needle hub (50) into inner tube (20). With the engagement between the flexible tabs (32) and the stop (23), the outer needle (52) is securely held inside but is not escaped from the barrel (10). Therefore, a user can protect him from being pricked by a used outer needle (52), and the operation of the blood sampling device in accordance with the present invention is easy and conveniently.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A blood sampling device comprising
a barrel being hollow and comprising
an inner surface;
a front end being closed and comprising an inner surface;
a rear end;
a through hole defined through the front end;
a pipe formed on the inner surface of the front end and comprising a free end;
at least one rib formed on the inner surface near the front end;
an opening defined on the rear end; and
a cap extending from the opening and comprising
an inner surface;
a circumference; and
at least one protrusion formed along the circumference and on the inner surface;
an inner tube mounted in the barrel and comprising
a front end;
a rear end;
an inner surface; and
an annular flange radially protruding on the front end and comprising a bottom abutting with the at least one rib;
a base being cylindrical, mounted in the inner tube, and comprising
a center;
an outer circumference;
an axis;
an upper portion;
an lower portion;
a center through hole defined in the center of the base;
at least one tab protruding on the outer circumference; and
multiple flexible tabs extending from the lower portion and selectively abutting with the annular flange; and
a spring mounted around the upper portion of the base and comprising
two ends, wherein one end abuts against the inner surface of the front end of the barrel and the other end abuts against the tab of the base;
a needle hub mounted in the base and comprising
an inner needle passing through the center through hole of the base; and
an outer needle passing through the through hole of the barrel.

2. The blood sampling device as claimed in claim 1, wherein
the pipe comprises multiple notches defined in the free end; and
the flexible tabs correspond respectively to the notches.

3. The blood sampling device as claimed in claim 2, wherein the inner tube comprises multiple longitudinal grooves defined through the inner tube.

4. The blood sampling device as claimed in claim 3, wherein the inner tube comprises a stop protruding on the inner surface of the inner tube near the rear end.

5. The blood sampling device as claimed in claim 4, wherein the barrel comprises two finger flanges respectively extending from the rear end.

6. The blood sampling device as claimed in claim 5, wherein the barrel has multiple ribs corresponding to each other.

7. A barrel assembly for blood sampling device comprising
a barrel being hollow and comprising
an inner surface;
a front end being closed and comprising an inner surface;
a rear end;
a through hole defined through the front end;
a pipe formed on the inner surface of the front end and comprising a free end;
at least one rib formed on the inner surface near the front end;
an opening defined on the rear end; and
a cap extending from the opening and comprising
an inner surface;
a circumference; and
at least one protrusion formed along the circumference and on the inner surface;
an inner tube mounted in the barrel and comprising
a front end;
a rear end;
an inner surface; and
an annular flange radially protruding on the front end and comprising a bottom abutting with the at least one rib;
a base being cylindrical, mounted in the inner tube, and comprising
a center;
an outer circumference;
an axis;
an upper portion;
an lower portion;
a center through hole defined in the center of the base;
at least one tab protruding on the outer circumference; and
multiple flexible tabs extending from the lower portion and selectively abutting with the annular flange; and
a spring mounted around the upper portion of the base and comprising
two ends, wherein one end abuts against the inner surface of the front end of the barrel and the other end abuts against the tab of the base.

8. The barrel assembly for blood sampling device as claimed in claim 7, wherein
the pipe comprises multiple notches defined in the free end; and
the flexible tabs correspond respectively to the notches.

9. The barrel assembly for blood sampling device as claimed in claim 8, wherein the inner tube comprises multiple longitudinal grooves defined through the inner tube.

10. The barrel assembly for blood sampling device as claimed in claim 9, wherein the inner tube comprises a stop protruding on the inner surface of the inner tube near the rear end.

11. The barrel assembly for blood sampling device as claimed in claim 10, wherein the barrel comprises two finger flanges respectively extending from the rear end.

12. The barrel assembly for blood sampling device as claimed in claim 11, wherein the barrel has multiple ribs corresponding to each other.
